(19) United States
(12) Patent Application Publication
(10) Pub. No.: US 2008/0300684 A1
(43) Pub. Date: Dec. 4, 2008

(54) SYSTEM AND METHOD FOR CONTROLLED DELIVERY OF BONE MORPHOGENIC PROTEIN AND OTHER BONE GRAFT SUBSTITUTES FOR BONE FORMATION, REPAIR AND HEALING

(76) Inventor: Alexis P. Shelokov, Dallas, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(21) Appl. No.: 11/756,223

(22) Filed: May 31, 2007

(51) Int. Cl. A61F 2/28

(52) U.S. Cl. 623/16.11

(57) ABSTRACT

An implant system delivers bone morphogenic protein (BMP) and other substances to the human body. The implant system includes an intake module for receiving the BMP and other substances. The intake module includes a reservoir implanted at least partially in the body. The implant system includes a delivery module implanted in the human body and configured to administer the BMP and other substances. The delivery module is connected to the intake module through one or more tubes implanted in the human body. The tubes transport the BMP and other substances from the intake module to the delivery module.

### FIELD OF THE INVENTION

The invention relates to systems and methods for delivering bone morphogenic protein (BMP) and other bone graft substitutes. More specifically, the invention relates to a system and method for controlled delivery of BMP and other bone graft substitutes to aid in bone formation, repair and healing.

### BACKGROUND OF THE INVENTION

BMP and other bone graft substitutes are used by surgeons to fill in empty space created in or between the bones of the spine by disease, injury, deformity or during a surgical procedure such as spinal fusion. These bone graft substitutes stimulate bone healing and provide a biologically compatible framework for new bone to grow into.

BMPs are produced in the human body and regulate bone formation and healing. They can speed up healing and lessen negative reaction to bone substitutes. They can be extracted from human or cow bones, and can also be produced in laboratories. It has been generally shown that about 1 gm of bone is produced for each milligram of BMP implanted. BMP is available in the form of a powder, crystal, or liquid.

Demineralized Bone Matrix (DBM) is a bone substitute that is a product of processed allograft bone. DBM contains collagen, proteins and growth factors that are extracted from the allograft bone. DBM is available in the form of a powder, crushed granules or as a gel.

Ceramics are also used as bone substitutes. The are available in many forms such as porous and mesh. Ceramics provide a framework for bone growth, but do not contain natural proteins that influence bone growth.

Current techniques allow delivery of BMP and other bone substitutes during surgery. If the bone does not heal or repair adequately, it may be necessary to administer additional doses of BMP or other bone substitutes at a later time, sometimes several weeks after the initial surgery. It may also be advantageous to administer bone marrow and bone marrow aspirates to a fusion site in addition to the BMP and other substitutes. In order to administer additional doses of BMP or other substitutes, a patient must undergo another surgery in order to access the bone or the fusion site to administer the BMP or other substitutes.

### SUMMARY OF THE INVENTION

An implant system delivers bone morphogenic protein (BMP) and other substances to the human body. The implant system includes an intake module for receiving the BMP and other substances. The intake module includes a reservoir implanted at least partially in the body. The implant system includes a delivery module implanted in the human body and configured to administer the BMP and other substances. The delivery module is connected to the intake module through one or more tubes implanted in the human body. The tubes transport the BMP and other substances from the intake module to the delivery module. The delivery module may be a fenestrated tube configured to administer the BMP or other substances to the human body. The delivery module can have any suitable shape and may have holes or openings to release the BMP or other substances. The BMP may be in a liquid or a gel form so that it can be injected into the intake module by a syringe.

### BRIEF DESCRIPTION OF THE DRAWINGS

For a more complete understanding of the features and advantages of the present invention, reference is now made to the detailed description of the invention along with the accompanying figures and in which:

FIG. 1A illustrates an exemplary embodiment of an implant system for delivery of bone morphogenic protein (BMP) and other substances to the human body.

FIG. 1B illustrates an exemplary embodiment of a delivery module.

FIG. 2 illustrates an exemplary embodiment of an implant system implanted in the human body.

FIG. 3 shows an intake module implanted beneath a skin.

FIGS. 4A and 4B illustrate other embodiments of an implant for use in the human body.

FIG. 5 illustrates a multilayered BMP (or other substances).

FIGS. 6A and 6B show another embodiment of an implant system.

### DETAILED DESCRIPTION OF THE EMBODIMENTS

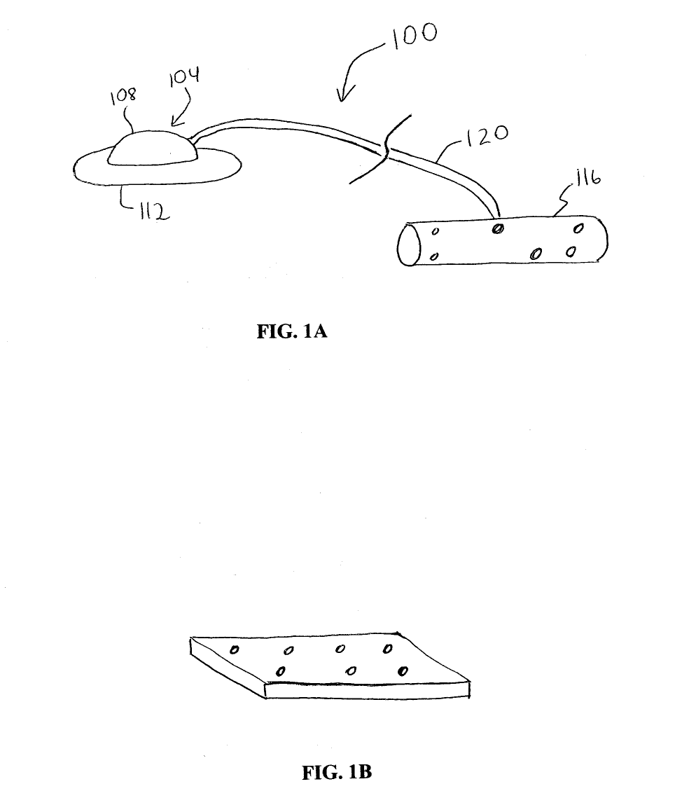

FIG. 1A illustrates an exemplary embodiment of an implant system 100 for delivery of bone morphogenic protein (BMP), bone graft substitutes and other substances such as bone marrow, bone marrow aspirates, allograft and autograft bone (hereinafter referred to collectively as “BMP and other substances”) to the human body. The implant system 100 includes an intake module 104. In one embodiment, the intake module 100 is a port adapted to receive BMP and other substances via a syringe.

The intake module 104 includes a reservoir 108 or other mechanism suitable for receiving BMP and other substances. The reservoir 108 is supported by a base 112. The intake module 104 may be fabricated of a flexible, biocompatible or subdermal implant material, such as Silastic brand silicone rubber and similar polymers. The intake module 104 is implanted in the human body beneath the skin and is accessible from outside through a needle or other means.

The implant system 100 also includes a delivery module 116. The delivery module 116 is coupled to the intake module via a tube 120. The tube 120 transports BMP and other substances from the intake module 104 to the delivery module 116. The tube 120 and the delivery module 116 are both implanted in the human body, with the delivery module 116 being implanted near the bone where fusion is desired or any other location where BMP and other substances will be delivered.

In one embodiment, two or more delivery modules 116 may be coupled to a single intake module 104 through a plurality of tubes 120. The tube 120 is made from a flexible material safe and suitable for implantation in the human body. The delivery module 116 may be fabricated of a flexible, biocompatible or subdermal implant material, such as Silastic brand silicone rubber and similar polymers.

The delivery module 116 may take the form of a cylinder or a strip adapted to receive BMP and other substances from the intake module 104. The delivery module 116 may be fenestrated (or perforated or may have a plurality of openings) for the release of BMP and other substances into the human body. The delivery module 116 may have other means suitable for the release of BMP. The delivery module 116 may have any other shape (e.g., ravioli-shaped) suitable for implantation in the human body. In one exemplary embodiment shown in FIG. 1B, the delivery module may take the shape of a strip which is between 1 to 1.5 cm wide and between 0.4 to 4 mm thick.

The delivery module may be placed on a graft bed or a fusion bed adjacent to a spinal implant or where fusion is desired. The graft bed is created from a graft material. The delivery module releases the BMP and other substances to induce healing, fusion or formation of bone. The intake module 104 preferably receives BMP and other substances in a liquid, gel or fluid-like form. As discussed before, the BMP and other substances are injected into the intake module 104 by a syringe. As the BMP and other substances are injected to the intake module 104, the pressure from the syringe forces the BMP and other substances to flow into the delivery module 116. The pressure from the syringe also forces the BMP and other substances to flow out of the delivery module 116 through holes or openings formed on the delivery module 116. The delivery module may be placed on a fusion bed. The fusion bed may be filled with autograft or allograft.

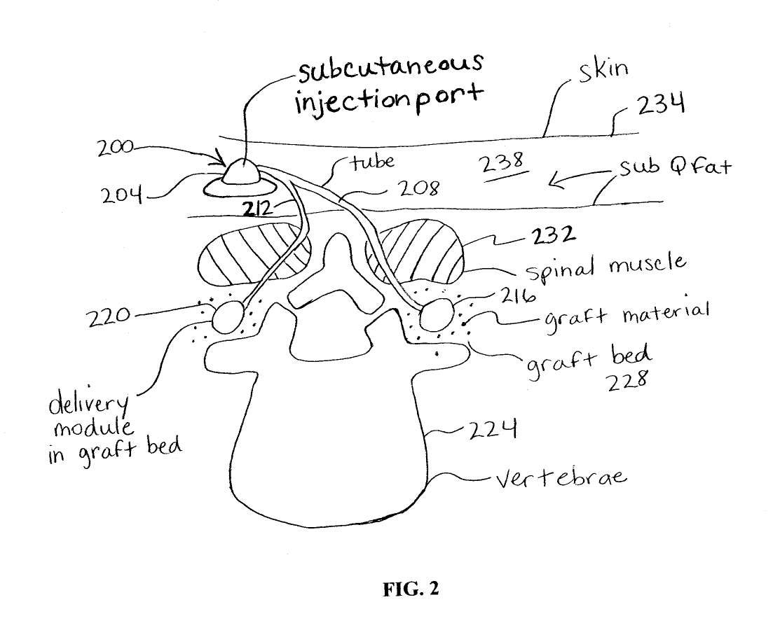

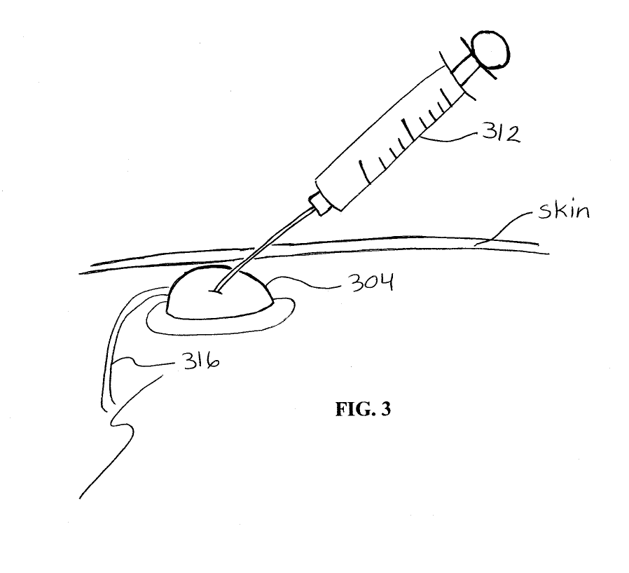

FIG. 2 illustrates an exemplary embodiment of an implant system 200, which is implanted in the human body for the delivery of BMP and other substances to a vertebrae for inducement of bone formation, fusion or healing. The implant system 200 includes an intake module 204 coupled to two delivery modules 216 and 220 via tubes 208 and 212, respectively. The delivery modules 216 and 220 are placed laterally adjacent to the vertebrae, one delivery module on each side of the vertebrae. The intake module 204 is implanted in the subcutaneous region 238 beneath the skin 234. The tubes 208 and 212 transport BMP and other substances from the intake module 204 to the delivery modules 216 and 220. As shown in FIG. 2, the tubes 208 and 212 are implanted through the spinal muscle 232 to supply BMP and other substances to the delivery modules 216 and 220. FIG. 3 shows an intake module 304, which is implanted beneath a skin 308, receiving BMP and other substances through a syringe 312. The pressure from the syringe 312 forces BMP and other substances to flow to a delivery module and finally out of the delivery module through holes or openings.

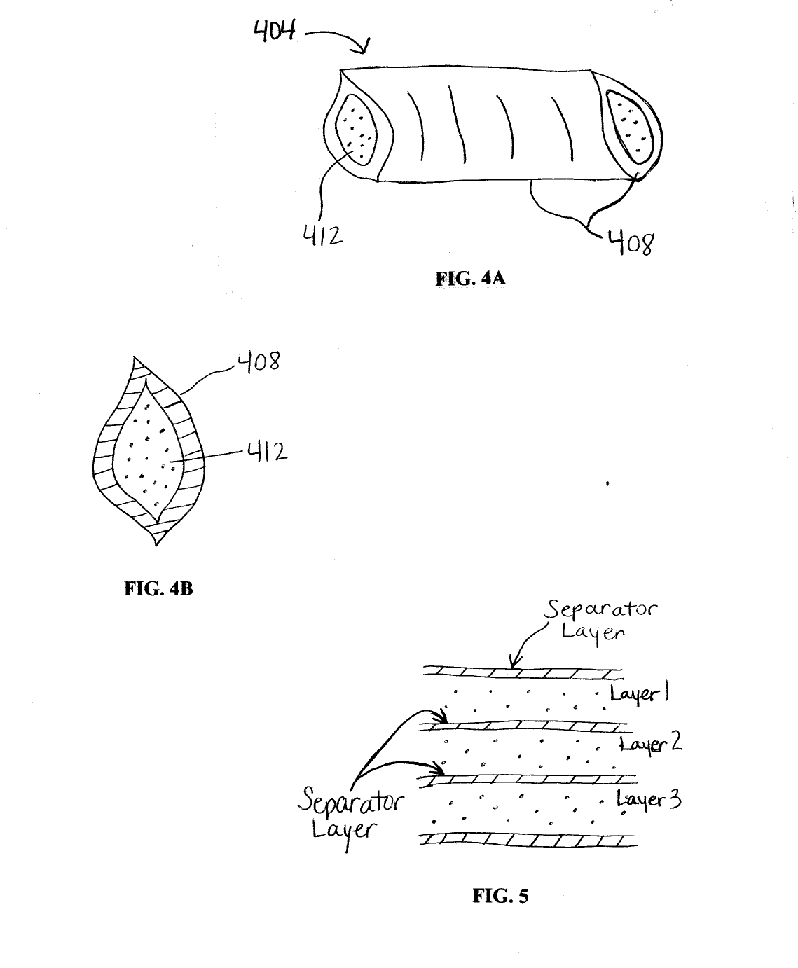

FIGS. 4A and 4B illustrate another exemplary embodiment of an implant for use in the human body for the controlled release of BMP and other substances. The implant is a delivery module 404 formed by one or more external layers 408 of bioabsorbable material defining a housing space. A predetermined amount of BMP and/or other substances 412 are placed in the housing space. The release of BMP into the human body can be controlled or delayed by varying the thickness of the external layers 408 formed by the bioabsorbable material. For a quick release of BMP, a relatively thin external layer 408 can be used, while for a delayed release of BMP, a relatively thick external layer 408 can be used. As noted before, the external layer of bioabsorbable material can be formed by a single layer or can be formed by multiple layers (i.e., lamination) of bioabsorbable material. In one exemplary embodiment, the delivery module may take the shape of a strip or a ravioli, which is between 1 to 1.5 cm wide and between 0.4 to 4 mm thick.

As the external layer 408 is absorbed or degrades into the human body, BMP and/or other substances are released, which induces the formation, fusion or healing of the bone such as, for example, a vertebrae.

In one exemplary embodiment, BMP powder or gel can be incorporated into layers of polyglycolic or polylactic acid and made into sheets. Alternatively, sheets may be made by BMP or other substances, which are then annealed to each other with heat or collagen glue. One or more layers of BMP can be placed inside the delivery module 404. As the external layer 408 is absorbed or degrades into the human body, increasing layers of BMP will be exposed, thus delivering BMP over a period of time. As such, controlled or delayed delivery of BMP or other substances can be achieved. The other substances may include DBM, allograft or bone marrow. The layers of BMP or other substances may be separated by one or more separator layers formed by, for example, bioabsorbable material. FIG. 5 illustrates a multilayered BMP or other substances comprising layers 1-3. Each layer, for example, can be formed using the same type of BMP or a different type of BMP. Thus, for example, layer 1 may be formed with BMP 7, layer 2 may be formed with BMP 9, and layer 3 may be formed with BMP 11. Alternately, each layer can have a combination of two or more types of BMP or other substances.

The multilayered embodiment shown in FIG. 5 allows each layer to be exposed to body surfaces over a period of time. For example, the thickness and the types of BMP in each layer in FIG. 5 can be adjusted so that layer 1 is exposed for a time period T1, after which layer 2 is exposed for a time period T2, and after which layer 3 is exposed for a time period T3. The time periods may vary from a few days to a few weeks depending on the type and thickness of the layers. In another exemplary embodiment, one or more layers may have allograft bone (i.e., cadaver bone) or DBM for enhanced healing or repair.

The delivery module 404 can be implanted in a fusion bed during a surgery. The fusion bed may be filled with autograft, that is, bone from the patient, or allograft, that is, bone from a cadaver.

In one embodiment, the external layers 408 of the delivery module 404 can be made from a flexible, soft material such as cylastic or other material suitable for implantation. In other words, the external layers 408 can be made from materials that are not bio-absorbable. If cylastic or other non-bioabsorbable materials are used to make the external layers 408, holes or other openings are formed on the external layers 408 to allow the BMP and other substances to flow out of the delivery module 404. BMP and/or other substances inside the delivery module 404 can be in a powdered form, or in a crystalline form. Also, BMP and/or other substances can be in one or more layers.

The delivery module 404 allows the delivery of BMP and other substances to the desired area at an advantageous time for fusion, healing or bone formation. For example, the implant system shown in FIG. 1 allows the delivery of BMP and other substances a few days or a few weeks after the surgery. Also, different types of BMP can be injected for fusion or healing at desired times.

It will be apparent that the implant systems discussed herein can be used to inject stem cells, bone marrow, antibiotics and other bone graft substitutes and other substances in addition to BMP at desired times. For example, stem cells, bone marrow, antibiotics and/or other substances can be added to the BMP layers 1-3 shown in FIG. 5.

The bioabsorbable material may be a polylactic acid, a polyglycolic acid, or any other suitable bioabsorbable material. In one exemplary embodiment, the BMP and/or other substances may be contained in a physiologically acceptable, biodegradable, porous ceramic. The porous ceramic containing BMP may then be placed inside the delivery module 404 shown in FIGS. 4A and 4B. Also, BMP or other substances may be contained in triphosphates or other suitable materials and placed inside the delivery module 404.

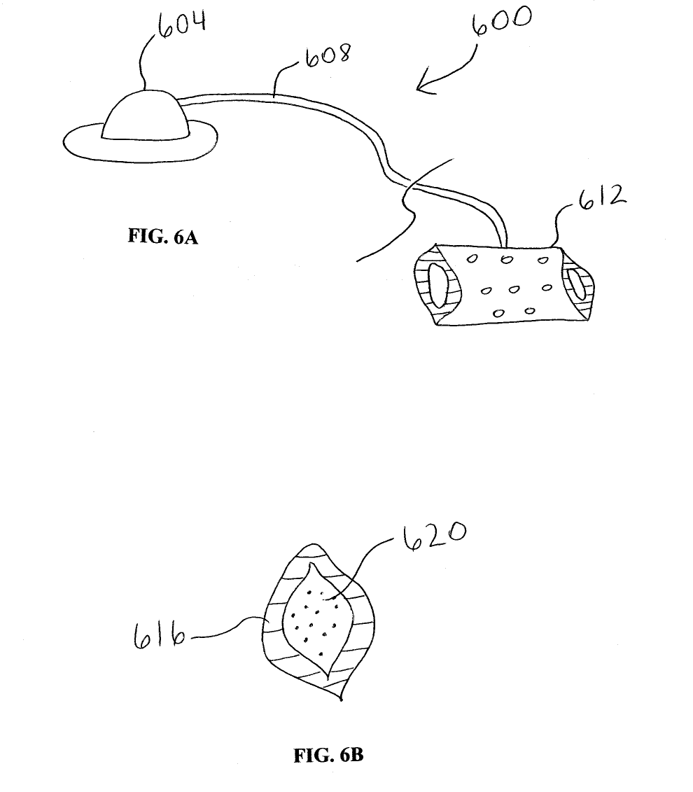

FIGS. 6A and 6B show an exemplary implant system 600 that combines some of the elements of the embodiments shown in FIGS. 1 and 4A. The implant system 600 includes an intake module 604 adapted to receive BMP or other substances preferably by a syringe. A tube 608 transports BMP and other substances from the intake module 604 to a delivery module 612. The delivery module 612 includes a housing to retain one or more layers of BMP or other substances. The layers may be separated by one or more separator layers as discussed in connection with FIGS. 4A, 4B and 5. FIG. 6B is a cross sectional view of the delivery module 612. The delivery module includes an external layer 616 defining a housing space 620. The housing space 620 is adapted to contain a predetermined amount of BMP. The delivery module 620 is also adapted to receive additional BMP from the intake module. The delivery module 620 may be fenestrated or may include perforations to release BMP and other substances to the tissue for bone formation, repair or fusion.

The delivery module 612 includes a predetermined amount of BMP or other substances, but is also capable of receiving additional BMP or other substances from the intake module 614. The BMP or other substances inside the delivery module 612 may be in one or more layers or may be in other form. In one embodiment, the external layer 616 of the delivery module 612 is preferably made from a bioabsorbable material. Initially, the delivery module 612 releases BMP or other substances supplied by the intake module 614 via the tube 608. The BMP or other substances flow out into the human body through the holes or fenestrations on the external layer 616. Eventually, the external layer 616 degrades into the human body exposing the substances (i.e., BMP and/or other substances) contained inside. The delivery module 612 may also contain bone, bone marrow or other substances in addition to BMP.

The implant system 600 allows the delivery of BMP or other substances to a tissue at the time of surgery or thereafter. Also, when the external layer 616 degrades, layers of BMP and/or other substances are exposed to the wound inducing healing and/or fusion. The implant system 600 can be used to accelerate the fusion process with additional BMP, bone marrow or stem cells of various origins.

In one exemplary embodiment, one or more antibiotics may be added to BMP. Thus, for example, one or more antibiotics may be added to BMP contained inside the delivery modules 404 and 612 discussed above. Also, one or more antibiotics can be delivered to bone or other tissue using the implant system shown in FIG. 1. As will be appreciated by those skilled in the art, infection often occurs in spinal fusion and other bone surgeries. The exemplary embodiments can be used to deliver antibiotics in a sustained or controlled manner.

While the compositions, structures, apparatus and methods of this invention have been described in terms of preferred embodiments, it will be apparent to those of skill in the art that variations may be applied to the compositions, structures, apparatus and/or methods and in the steps or in the sequence of steps of the method described herein without departing from the concept, spirit and scope of the invention. For example, it will be apparent to those skilled in the art that the dimensions of the various modules and their components disclosed in the foregoing detailed description, including length, width, thickness of the various modules such as the intake modules, the delivery modules, the tubes, and the various layers of BMP and other substances can be any suitable dimensions. All such substitutes and modifications apparent to those skilled in the art are deemed to be within the spirit, scope and concept of the invention as defined by the appended claims.

What is claimed is:

1. An implant system for delivery of bone morphogenic protein (BMP) or other substances to the human body, comprising:
an intake module for receiving the BMP or other substances;
a delivery module implanted in the human body and configured to administer the BMP or other substances; and
one or more tubes implanted in the human body and adapted to transport the BMP or other substances from the intake module to the delivery module.

2. The implant system of claim 1, wherein the intake module further comprises:
a reservoir implanted at least partially inside the body, the reservoir configured to store the BMP or other substances prior to supplying to the delivery module through the tubes, the reservoir having at least one intake port for receiving the BMP or other substances.

3. The implant system of claim 1, wherein the delivery module is implanted in a graft bed in proximity to a bone.

4. The implant system of claim 1, wherein the delivery module is a fenestrated tube configured to administer the BMP or other substances to the human body.

5. The implant system of claim 1, wherein the delivery module comprises a plurality of openings to deliver the BMP or other substances to tissues.

6. The implant system of claim 1, wherein the BMP is in a liquid form.

7. The implant system of claim 1, wherein the BMP is in a gel or fluid form.

8. The implant system of claim 1, wherein the intake module receives the BMP or other substances from a syringe.

9. The implant system of claim 1, wherein the BMP induces spinal fusion.

10. An implant for use in the human body for the controlled release of bone morphogenic protein (BMP) or other substances for inducing bone formation or healing comprising:
a delivery module formed by at least one external layer of bioabsorbable material defining a housing space; and
a predetermined amount of BMP or other substances placed in the housing space, wherein at least a portion of the external layer of bioabsorbable material is absorbed into the human body thereby causing the release of the BMP or other substances.

11. The implant of claim 10, wherein the bioabsorbable material is a polylactic acid.

12. The implant of claim 10, wherein the bioabsorbable material is a polyglycolic acid.

13. The implant of claim 10, wherein the release of BMP or other substances induces new bone formation.

14. The implant of claim 10 further comprising a predetermined amount of allograft bone placed inside the delivery module.

15. The implant of claim 10 further comprising at least one layer of allograft bone separated from the BMP by at least one internal layer of bioabsorbable material, wherein the allograft bone layer and the BMP is enclosed by the external layer of bioabsorbable material.

16. The implant of claim 10, wherein the BMP is formed as a plurality of layers inside the delivery module.

17. The implant of claim 16, wherein the layers are separated from one another by a bioabsorbable separator layer.

18. The implant of claim 10, wherein the BMP is in a powdered form.

19. The implant of claim 10, wherein the BMP is in crystalline form.

20. A multi-layered implant for use in the human body for controlled release of bone morphogenic protein (BMP) or other substances for inducing bone formation or healing, comprising:
a delivery module formed by at least one external layer of bioabsorbable material and defining a housing space; and
a plurality of layers of BMP or other substances placed in the housing space, the layers separated by at least one separator layer, wherein at least a portion of the external layer of bioabsorbable material is absorbed into the human body thereby causing the release of BMP and other substances.

21. The multi-layered implant of claim 20, wherein the separator layers are formed by the bioabsorbable material.

22. The multi-layered implant of claim 20, wherein each layer of BMP is made from one or more selected types of BMP.

23. The multi-layered implant of claim 22, wherein the layers of BMP vary from one to another due to the type of BMP in the layers.

24. The multi-layered implant of claim 20, wherein different layers of BMP are made from different type of BMP.

25. The multi-layered implant of claim 20, wherein the bioabsorbable material is a polylactic acid.

26. The multi-layered implant of claim 20, wherein the bioabsorbable material is a polyglycolic acid.

27. The multi-layered implant of claim 20 further comprising at least one layer of allograft bone placed inside the delivery module.

28. The multi-layered implant of claim 20, wherein the BMP is in a powdered form.

29. The multi-layered implant of claim 20, wherein the BMP is in a crystalline form.

30. A multi-layered implant for use in the human body for controlled release of bone morphogenic protein (BMP) or other substances for inducing bone formation, comprising:
a plurality of layers of BMP or other substances each separated by at least one internal layer of bioabsorbable material; and
an external layer of the bioabsorbable material enclosing the layers of BMP or other substances and the internal layer of bioabsorbable material, wherein at least a portion of the external layer of bioabsorbable material is absorbed into the human body thereby causing the release of the BMP or other substances, and the layers of BMP vary from one another due to the type of BMP in the layers.

31. The multi-layered implant of claim 30, wherein the different layers of the BMP are made from different type of BMP.

32. The multi-layered implant of claim 30, wherein the layers of BMP vary from one another due to the type of BMP in the layers.

33. An implant system for use in the human body for the delivery of bone morphogenic protein (BMP) or other substances for inducing bone formation, comprising:
an intake module for receiving the BMP or other substances;
a delivery module implanted in the human body and configured to store and release the BMP or other substances into the human body, the delivery module also configured to receive the BMP or other substances from the intake module; and
one or more tubes implanted at least partially in the human body and adapted to transport the BMP or other substances from the intake module to the delivery module.

34. The implant system of claim 33, wherein the delivery module further comprises:
a predetermined amount of the BMP or other substances covered with one or more external layers of bioabsorbable materials; wherein at least a portion of the external layers are absorbed into the human body thereby causing the release of the BMP or other substances.

35. The implant system of claim 33, wherein the delivery module further comprises:
a plurality of layers of BMP or other substances placed in the housing space, the layers of BMP or other substances each separated by at least one separator layer,
an external layer of the bioabsorbable material enclosing the layers of BMP or other substances and the internal layer of bioabsorbable material, wherein at least a portion of the external layer is absorbed into the human body thereby causing the release of the BMP or other substances, and the layers of BMP or other substances vary from one another due to the type of BMP in the layers.

36. The multi-layered implant of claim 33, wherein the separator layers are formed by the bioabsorbable material.

37. The multi-layered implant of claim 33, wherein each layer of BMP is made from one or more selected types of BMP.

38. The multi-layered implant of claim 33, wherein the layers of BMP vary from one another due to the type of BMP in the layers.

39. The multi-layered implant of claim 33, wherein different layers of the BMP are made from different type of BMP.

40. An implant for use in the human body for the controlled release of bone morphogenic protein (BMP) or other substances for inducing bone formation or healing comprising:
a delivery module formed by at least one external layer defining a housing space, the delivery module having a one or more holes or openings; and
a predetermined amount of BMP or other substances placed in the housing space, wherein the BMP or other substances is released out of the delivery module through the holes or openings into the human body.

41. The implant of claim 40, wherein the external layer is formed by a flexible material.

42. The implant of claim 40, wherein the external layer is formed by cylastic.

43. An implant system for delivery of bone morphogenic protein (BMP) for spinal fusion, comprising:
an intake module for receiving the BMP;
a delivery module implanted in the human body and configured to administer the BMP, the delivery module having one or more holes or openings; and
one or more tubes implanted in the human body and adapted to transport the BMP from the intake module to the delivery module, wherein the BMP is released out of the delivery module through the holes or the openings causing spinal fusion.
